(19) United States
(12) Patent Application Publication
(10) Pub. No.: US 2025/0388820 A1
(43) Pub. Date: Dec. 25, 2025

(54) RECYCLE OF C4- OR C6- LIGHT HYDROCARBON LIQUIDS TO ACID CONDENSATION FEED

(71) Applicants: Johnson Matthey Public Limited Company, London (GB); Virent, Inc., Madison, WI (US)

(72) Inventors: Ian Campbell, London (GB); Rauf Edward John Gearing, London (GB); Matthew Van Straten, Madison, WI (US); Andrew Held, Madison, WI (US)

(21) Appl. No.: 19/244,629

(22) Filed: Jun. 20, 2025

Related U.S. Application Data
(60) Provisional application No. 63/662,811, filed on Jun. 21, 2024.

(51) Int. Cl. C10G 3/00; C10G 7/08

(52) U.S. Cl. CPC C10G 3/44; C10G 7/08; C10G 2300/1011; C10G 2300/4081; C10G 2300/70

(57) ABSTRACT

The present disclosure provides methods and systems for producing a C₄₊ compound, including the steps of: (i) reacting a feed stream comprising C₁₊O₁₋₃ hydrocarbons in the presence of an acid condensation (AC) catalyst at a condensation temperature and condensation pressure to produce an AC product stream including the C₄₊ compound; (ii) fractionating AC product stream into an AC liquid product stream including organic products and a vapor stream; (iii) fractionating the AC liquid product stream by a fractionation column into a liquid C₄₊ compound product stream and a gaseous overhead stream; (iv) cooling the gaseous overhead stream to produce a liquid reflux stream and a gaseous vent stream; (v) recycling a first portion of the liquid reflux stream to the fractionation column for the fractionation in step (iii); and (vi) recycling a second portion of the liquid reflux stream to the feed stream for the reaction in step (i).

### CROSS REFERENCE TO RELATED APPLICATIONS

This application claims the benefit of priority to U.S. Provisional Application No. 63/662,811, filed Jun. 21, 2024, the entire contents of which is incorporated by reference herein.

### BACKGROUND

Significant amount of attention has been placed on developing new technologies for more efficient energy production. Bioreforming processes can produce aromatic hydrocarbons and other useful compounds from biomass feedstocks, including cellulose, hemicellulose, and lignin. For instance, cellulose and hemicellulose can be used as feedstock for various bioreforming processes, including aqueous phase reforming (APR) and hydrodeoxygenation (HDO)—catalytic reforming processes that, when integrated with hydrogenation, can convert cellulose and hemicellulose into an array of products, including hydrogen, liquid fuels, aromatics, kerosene, diesel fuel, lubricants, and fuel oils, among others. In addition, catalytic acid condensation (AC) can be used to convert oxygenates (e.g., generated by HDO) or other compounds into hydrocarbons.

APR and HDO methods and techniques are described in U.S. Pat. Nos. 6,699,457; 6,964,757; 6,964,758; and 7,618,612 (all to Cortright et al., entitled “Low-Temperature Hydrogen Production from Oxygenated Hydrocarbons”); U.S. Pat. No. 6,953,873 (to Cortright et al., entitled “Low-Temperature Hydrocarbon Production from Oxygenated Hydrocarbons”); and U.S. Pat. Nos. 7,767,867; 7,989,664;; and 8,198,486 (all to Cortright, entitled “Methods and Systems for Generating Polyols”), all of which are incorporated herein by reference. Various other APR and HDO methods and techniques are also described in U.S. Pat. Nos. 8,053,615; 8,017,818; 7,977,517; 8,362,307; 8,367,882; and 8,455,705 (all to Cortright and Blommel, entitled “Synthesis of Liquid Fuels and Chemicals from Oxygenated Hydrocarbons”); U.S. Pat. No. 8,231,857 (to Cortright, and entitled “Catalysts and Methods for Reforming Oxygenated Compounds”); U.S. Pat. No. 8,350,108 (to Cortright et al., entitled “Synthesis of Liquid Fuels from Biomass”); and International Patent Application No. PCT/US2008/056330 (to Cortright and Blommel, entitled “Synthesis of Liquid Fuels and Chemicals from Oxygenated Hydrocarbons” and published as WO2008109877A1), all of which are incorporated herein by reference.

Accordingly, there is a need for methods for efficiently recycling hydrocarbons produced during bioreforming processes.

### SUMMARY OF THE INVENTION

Some aspects of the present disclosure provide a method of producing a C₄₊ compound, including the steps of: (i) reacting a feed stream comprising C₁₊O₁₋₃ hydrocarbons in the presence of an acid condensation (AC) catalyst at a condensation temperature and condensation pressure to produce an AC product stream including the C₄₊ compound; (ii) fractionating AC product stream into an AC liquid product stream including organic products and a vapor stream; (iii) fractionating the AC liquid product stream by a fractionation column into a liquid C₄₊ compound product stream and a gaseous overhead stream; (iv) cooling the gaseous overhead stream to produce a liquid reflux stream and a gaseous vent stream; (v) recycling a first portion of the liquid reflux stream to the fractionation column for the fractionation in step (iii); and (vi) recycling a second portion of the liquid reflux stream to the feed stream for the reaction in step (i).

Step (ii) can be carried out in a first phase separator.

Step (iv) can comprise: (iv-a) cooling the gaseous overhead stream by a condenser to produce a cooled overhead stream; and (iv-b) fractionating the cooled overhead stream in a second phase separator into the liquid reflux stream and the gaseous vent stream.

A first portion of the vapor stream of step (ii) can be recycled to the feed stream for the reaction in step (i). In some embodiments, the first portion of the vapor stream of step (ii) is combined with the second portion of the liquid reflux stream of step (vi) to form a combined recycle stream, which is recycled to the feed stream for the reaction in step (i).

A second portion of the vapor stream of step (ii) can be condensed to produce a recovered liquid stream. In some embodiments, the recovered liquid stream is combined with the AC liquid product stream of step (ii) to form a combined AC liquid product stream, which is fractionated in step (iii) to produce the liquid C₄₊ compound product stream and the gaseous overhead stream.

The ratio of the first portion of the liquid reflux stream of step (v) to the second portion of the liquid reflux stream of step (vi) is about 40:60 to about 60:40.

### BRIEF DESCRIPTION OF DRAWINGS

FIG. 1 shows a schematic illustration of a method in accordance with some embodiments of the present disclosure.

FIG. 2 shows yield (carbon percentage of feed) of C₄₊ and C₅₊ liquid products as a function of implementing C₄₋ and C₆₋ Light Hydrocarbon Recycle (LHR).

FIG. 3 shows the weight percent product species distribution for C₄₋ and C₆₋ Light Hydrocarbon Recycle (LHR).

### DETAILED DESCRIPTION OF THE INVENTION

The present disclosure relates to processes and systems for recycling C₄₋ or C₆₋ hydrocarbon products from acid condensation (AC) feed.

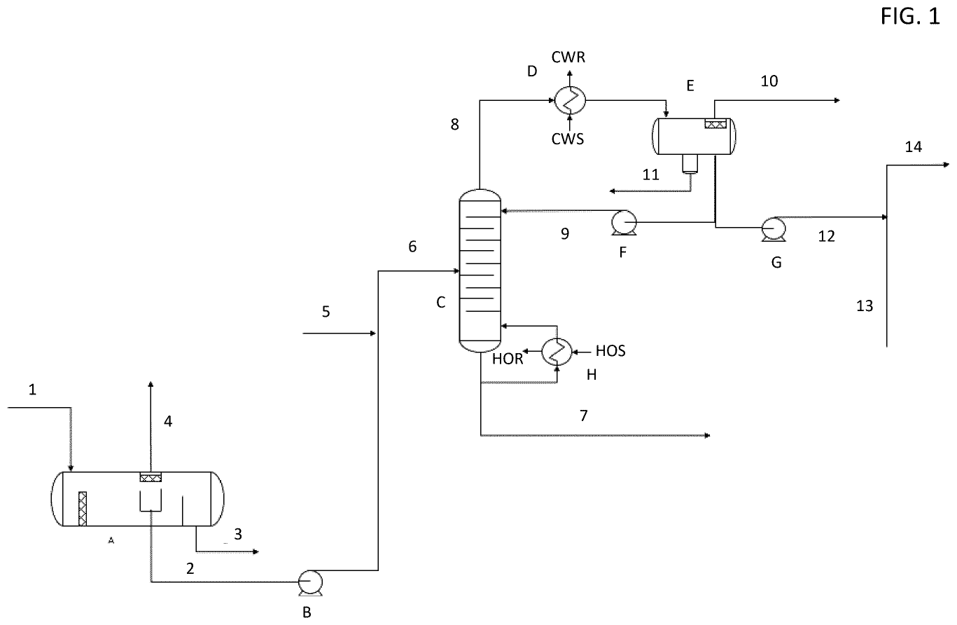

In one aspect, referring to FIG. 1, disclosed herein are methods of producing a C₄₊ compound, including the steps of: (i) reacting a feed stream comprising C₁₊O₁₋₃ hydrocarbons in the presence of an acid condensation (AC) catalyst at a condensation temperature and condensation pressure to produce an AC product stream comprising the C₄₊ compound (1), (ii) fractionating AC product stream into an AC liquid product stream (2) including organic products and a vapor stream (4); (iii) fractionating the AC liquid product stream (2) by a fractionation column (C) into a liquid C₄₊ compound product stream (7) and a gaseous overhead stream (8); (iv) cooling the gaseous overhead stream (8) to produce a liquid reflux stream (9) and a gaseous vent stream (10); (v) recycling a first portion of the liquid reflux stream (9) to the fractionation column (C) for the fractionation in step (iii); and (vi) recycling a second portion of the liquid reflux stream (12) to the feed stream for the reaction in step (i).

Generally, the technology disclosed herein can be used to improve AC processing for a wide range of feed streams. A wide variety of systems can be implemented to provide a feed stream to an AC reactor system (e.g., as variously disclosed in U.S. Pat. Nos. 6,699,457; 6,964,757; 6,964,758; 7,618,612; 6,953,873; 7,767,867; 7,989,664; 6,953,873; 7,767,867; 7,989,664; 8,198,486; 8,053,615; 8,017,818; 7,977,517; 8,362,307; 8,367,882; 8,455,705 8,231,857; and 8,350,108; in International Patent Publication WO2008109877A1; or as otherwise known in the art). In some cases, the aqueous feed stream is derived from biomass. In some cases, the feed stream may include C₁₊O₁₋₃ hydrocarbons.

In one aspect, the methods disclosed herein include fractionating AC product stream into an AC liquid product stream, including organic products, and a vapor stream. Following the AC reaction, the crude AC product can be fed to an AC product separator, which acts as a decanter to separate the water in the crude AC product from the organic components, which can then be fed to a column for fractionation into the AC liquid and vapor streams (e.g., a Lights Column). Referring to FIG. 1, the AC product separator (component A) may perform the fractionation to produce stream AC Organic product (2) and vapor stream (4). An aqueous product stream (3) can also be produced, which can be sent for organics recovery. Fractionation may be accomplished by any suitable separation methods and systems known in the art. In some cases, fractionation is carried out by a phase separator (i.e., a first phase separator). For example, the separator can be a 3-phase separator, which produces an aqueous liquid, an organic liquid, and a vapor phase. The liquid phases can be separated based on the different densities of the organics (less dense) and aqueous (more dense) liquid phases. In some cases, a first portion of the vapor stream (13) is recycled to the feed stream for the reaction in step (i), including reacting a feed stream comprising C₁₊O₁₋₃ hydrocarbons in the presence of an acid condensation (AC) catalyst at a condensation temperature and condensation pressure to produce an AC product stream comprising the C₄₊ compound (1). In some cases, the first portion of the vapor stream (13) is combined with the second portion of the liquid reflux stream (12) to form a combined recycle stream (14) which is recycled to the feed stream for the reaction in step (i), including reacting a feed stream comprising C₁₊O₁₋₃ hydrocarbons in the presence of an acid condensation (AC) catalyst.

In another aspect, a second portion of the vapor stream is condensed to produce a recovered liquid stream (5). In some cases, the recovered liquid stream (5) is combined with the AC liquid product stream (2) of step (ii) to form a combined AC liquid product stream (6), which is fractionated in step (iii) to produce the liquid C₄₊ compound product stream (7) and the gaseous overhead stream (8).

In another aspect, the methods disclosed herein include fractionating the AC liquid product stream (2) by a fractionation column (FIG. 1, component C, “Lights Column”) into a liquid C₄₊ compound product stream (7) and a gaseous overhead stream (8). Any suitable fractionation column may be used for the systems and methods disclosed herein, including packed columns, tray columns, structured columns, or spinning band columns.

In another aspect, the methods disclosed herein include cooling the gaseous overhead stream (8) to produce a liquid reflux stream (9) and a gaseous vent stream (10). Cooling may be accomplished by any suitable temperature control methods and systems known in the art. In some cases, cooling is carried out by a condenser. For example, a combination of a fin-fan air cooler and a water cooled trim condenser can be used. Referring to FIG. 1, in some cases, cooling the gaseous overhead stream (8) to produce a liquid reflux stream (9) and a gaseous vent stream (10) includes cooling the gaseous overhead stream (8) by a condenser (D) to produce a cooled overhead stream. The method may further include fractionating the cooled overhead stream in a second phase separator (e.g., a reflux drum, FIG. 1, component E) into the liquid reflux stream (9) and the gaseous vent stream (10). A produced water stream (11) can be removed from the second phase separator, which can be about 0.1% of the combined reflux and light hydrocarbon recycle (LHR) flow.

In another aspect, the methods disclosed herein include recycling a first portion of the liquid reflux stream (9) to the fractionation column (C) for the fractionation step (iii), wherein fractionating the AC liquid product stream (2) by a fractionation column (C) into a liquid C₄₊ compound product stream (7) and a gaseous overhead stream (8).

In some cases, the method may include recycling a second portion of the liquid reflux stream (12) to the feed stream for the reaction in step (i), including reacting a feed stream comprising C₁₊O₁₋₃ hydrocarbons in the presence of an acid condensation (AC) catalyst at a condensation temperature and condensation pressure to produce an AC product stream comprising the C₄₊ compound (1).

In another aspect, the ratio of the first portion of the liquid reflux stream (9) of step (v) to the second portion of the liquid reflux stream (12) of step (vi) is about 10:90 to about 90:10,about 20:80 to about 80:20, about 30:70 to about 70:30, about 40:60 to about 60:40, or about 50:50.

In some cases, the methods disclosed herein may further include isolating a liquid C₄₊ compound product stream (7). In some cases, the liquid C₄₊ compound product stream (7) has C₄₊ compounds at a yield of at least 80%, at least 75%, at least 70%, at least 65%, at least 60%, at least 55%, at least 50%, at least 45%, or at least 40%, based on total carbon amount of the feed stream; C₅₊ compounds at a yield of at least 80%, at least 75%, at least 70%, at least 65%, at least 60%, at least 55%, at least 50%, at least 45%, or at least 40%, based on total carbon amount of the feed stream; and at least 40%, at least 45%, at least 50%, at least 55%, at least 60%, at least 65%, at least 70%, at least 75%, at least 80%, at least 85%, or at least 90% aromatic compounds by weight. In some embodiments the liquid C₄₊ compound product stream has: C₄₊ compounds at a yield of at least 75% based on total carbon amount of the feed stream; C₅₊ compounds at a yield of at least 70% based on total carbon amount of the feed stream; and at least 50% aromatic compounds by weight. In some embodiments, the liquid C₄₊ compound product stream has: C₄₊ compounds at a yield of at least 70% based on total carbon amount of the feed stream; C₅₊ compounds at a yield of at least 65% based on total carbon amount of the feed stream; and at least 70% aromatic compounds by weight.

### AC Catalyst and AC Reactions

The methods and systems disclosed herein may be used for recovery of C₄₊ hydrocarbons produced from catalytic processes. As an example context, AC processing of hydrodeoxygenation (HDO) products, with the relevant HDO reactions being implemented for a feed stream from an upstream hydrogenation reactor system (not shown), are considered. A wide variety of systems can be implemented to provide a feed stream to an AC reactor system (e.g., as variously disclosed in U.S. Pat. Nos. 6,699,457; 6,964,757; 6,964,758; 7,618,612; 6,953,873; 7,767,867; 7,989,664; 6,953,873; 7,767,867; 7,989,664; 8,198,486; 8,053,615; 8,017,818; 7,977,517; 8,362,307; 8,367,882; 8,455,705 8,231,857; and 8,350,108; in International Patent Publication WO2008109877A1; or as otherwise known in the art). In some cases, the feed stream is derived from biomass. In some cases, the biomass feedstock includes cellulose, hemicellulose, and lignin. For instance, cellulose and hemicellulose.

In some examples, reacting the HDO product stream (or another product stream) in the presence of a condensation catalyst (i.e., in the AC reactor, D) can produce a C₄₊ compound. The C₄₊ compound can include a member selected from the group consisting of C₄₊ alcohol, C₄₊ ketone, C₄₊ alkane, C₄₊ alkene, C₅₊ cycloalkane, C₅₊ cycloalkene, aryl, fused aryl, and a mixture thereof. In one exemplary embodiment, the C₄₊ alkane comprises a branched or straight chain C₄₋₃₀ alkane, or a branched or straight chain C₄₋₉, C₇₋₁₄, C₁₂₋₂₄ alkane, or a mixture thereof. In another exemplary embodiment, the C₄₊ alkene comprises a branched or straight chain C₄₋₃₀ alkene, or a branched or straight chain C₄₋₉, C₇₋₁₄, C₁₂₋₂₄ alkene, or a mixture thereof. In another exemplary embodiment, the C₅₊ cycloalkane comprises a mono-substituted or multi-substituted C₅₊ cycloalkane, and at least one substituted group is a branched C₃₊ alkyl, a straight chain C₁₊ alkyl, a branched C₃₊ alkylene, a straight chain C₁₊ alkylene, a phenyl, or a combination thereof, or a branched C₃₋₁₂ alkyl, a straight chain C₁₋₁₂ alkyl, a branched C₃₋₁₂ alkylene, a straight chain C₁₋₁₂ alkylene, a phenyl, or a combination thereof, or a branched C₃₋₄ alkyl, a straight chain C₁₋₄ alkyl, a branched C₃₋₄ alkylene, straight chain C₁₋₄ alkylene, a phenyl, or a combination thereof. In another exemplary embodiment, the C₅₊ cycloalkene comprises a mono-substituted or multi-substituted C₅₊ cycloalkene, and at least one substituted group is a branched C₃₊ alkyl, a straight chain C₁₊ alkyl, a branched C₃₊ alkylene, a straight chain C₂₊ alkylene, a phenyl, or a combination thereof, or a branched C₃₋₁₂ alkyl, a straight chain C₁₋₁₂ alkyl, a branched C₃₋₁₂ alkylene, a straight chain C₂₋₁₂ alkylene, a phenyl, or a combination thereof, or a branched C₃₋₄ alkyl, a straight chain C₁₋₄ alkyl, a branched C₃₋₄ alkylene, straight chain C₂₋₄ alkylene, a phenyl, or a combination thereof. In another exemplary embodiment, the aryl comprises an unsubstituted aryl, or a mono-substituted or multi-substituted aryl, and at least one substituted group is a branched C₃₊ alkyl, a straight chain C₁₊ alkyl, a branched C₃₊ alkylene, a straight chain C₂₊ alkylene, a phenyl, or a combination thereof, or a branched C₃₋₁₂ alkyl, a straight chain C₁₋₁₂ alkyl, a branched C₃₋₁₂ alkylene, a straight chain C₂₋₁₂ alkylene, a phenyl, or a combination thereof, or a branched C₃₋₄ alkyl, a straight chain C₁₋₄ alkyl, a branched C₃₋₄ alkylene, a straight chain C₂₋₄ alkylene, a phenyl, or a combination thereof. In another exemplary embodiment, the fused aryl comprises an unsubstituted fused aryl, or a mono-substituted or multi-substituted fused aryl, and at least one substituted group is a branched C₃₊ alkyl, a straight chain C₁₊ alkyl, a branched C₃₊ alkylene, a straight chain C₂₊ alkylene, a phenyl, or a combination thereof, or a branched C₃₋₄ alkyl, a straight chain C₁₋₄ alkyl, a branched C₃₋₄ alkylene, a straight chain C₂₋₄ alkylene, a phenyl, or a combination thereof. In another exemplary embodiment, the C₄₊ alcohol comprises a compound according to the formula R¹—OH, wherein R¹ is a branched C₄₊ alkyl, straight chain C₄₊ alkyl, a branched C₄₊ alkylene, a straight chain C₄₊ alkylene, a substituted C₅₊ cycloalkane, an unsubstituted C₅₊ cycloalkane, a substituted C₅₊ cycloalkene, an unsubstituted C₅₊ cycloalkene, an aryl, a phenyl, or a combination thereof.

In another exemplary embodiment of method of making the C₄₊ compound, the C₄₊ ketone comprises a compound according to the formula

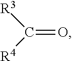

wherein R³ and R⁴ are independently a branched C₃₊ alkyl, a straight chain C₁₊ alkyl, a branched C₃₊ alkylene, a straight chain C₂₊ alkylene, a substituted C₅₊ cycloalkane, an unsubstituted C₅₊ cycloalkane, a substituted C₅₊ cycloalkene, an unsubstituted C₅₊ cycloalkene, an aryl, a phenyl, or a combination thereof. Examples of desirable C₄₊ ketones include, without limitation, butanone, pentanone, hexanone, heptanone, octanone, nonanone, decanone, undecanone, dodecanone, tridecanone, tetradecanone, pentadecanone, hexadecanone, heptyldecanone, octyldecanone, nonyldecanone, eicosanone, uneicosanone, doeicosanone, trieicosanone, tetraeicosanone, or isomers thereof.

The condensation catalyst is generally a catalyst capable of forming longer chain compounds by linking two molecules (e.g., oxygen containing species or other functionalized compounds, including olefins) through a new carbon-carbon bond, and converting the resulting compound to a hydrocarbon, alcohol, or ketone. In some embodiments, the condensation catalyst is an acid condensation catalyst. The condensation catalyst may include, without limitation, carbides, nitrides, zirconia, alumina, silica, aluminosilicates, phosphates, zeolites (e.g., ZSM-5,ZSM-11, ZSM-12, ZSM-22, ZSM-23, ZSM-35 and ZSM-48), titanium oxides, zinc oxides, vanadium oxides, lanthanum oxides, yttrium oxides, scandium oxides, magnesium oxides, cerium oxides, barium oxides, calcium oxides, hydroxides, heteropolyacids, inorganic acids, acid modified resins, base modified resins, and combinations thereof. The condensation catalyst may include the above alone or in combination with a modifier, such as Ce, La, Y, Sc, P, B, Bi, Li, Na, K, Rb, Cs, Mg, Ca, Sr, Ba, and combinations thereof. The condensation catalyst may also include a metal, such as Cu, Ag, Au, Pt, Ni, Fe, Co, Ru, Zn, Cd, Ga, In, Rh, Pd, Ir, Re, Mn, Cr, Mo, W, Sn, Os, alloys and combinations thereof, to provide a metal functionality.

The condensation catalyst may be self-supporting (i.e., the catalyst does not need another material to serve as a support) or may require a separate support suitable for suspending the catalyst in the reactant stream. One particularly beneficial support is silica, especially silica having a high surface area (greater than 100 square meters per gram), obtained by sol-gel synthesis, precipitation or fuming. In other embodiments, particularly when the condensation catalyst is a powder, the catalyst system may include a binder to assist in forming the catalyst into a desirable catalyst shape. Applicable forming processes include extrusion, pelletization, oil dropping, or other known processes. Zinc oxide, alumina, and a peptizing agent may also be mixed together and extruded to produce a formed material. After drying, this material is calcined at a temperature appropriate for formation of the catalytically active phase, which usually requires temperatures in excess of 450° C.

The condensation catalyst may include one or more zeolite structures comprising cage-like structures of silica-alumina. Zeolites are crystalline microporous materials with well-defined pore structures. Zeolites contain active sites, usually acid sites, which can be generated in the zeolite framework. The strength and concentration of the active sites can be tailored for particular applications. Examples of suitable zeolites for condensing secondary alcohols and alkanes may comprise aluminosilicates, optionally modified with cations, such as Ga, In, Zn, Mo, and mixtures of such cations, as described, for example, in U.S. Pat. No. 3,702,886, which is incorporated herein by reference. As recognized in the art, the structure of the particular zeolite or zeolites may be altered to provide different amounts of various hydrocarbon species in the product mixture. Depending on the structure of the zeolite catalyst, the product mixture may contain various amounts of aromatic and cyclic hydrocarbons.

Examples of suitable zeolite catalysts include ZSM-5, ZSM-11, ZSM-12, ZSM-22, ZSM-23, ZSM-35 and ZSM-48. Zeolite ZSM-5, and the conventional preparation thereof, is described in U.S. Pat. No. 3,702,886; Re. 29,948 (highly siliceous ZSM-5); U.S. Pat. Nos. 4,100,262 and 4,139,600, all incorporated herein by reference. Zeolite ZSM-11, and the conventional preparation thereof, is described in U.S. Pat. No. 3,709,979, which is also incorporated herein by reference. Zeolite ZSM-12, and the conventional preparation thereof, is described in U.S. Pat. No. 3,832,449, incorporated herein by reference. Zeolite ZSM-23, and the conventional preparation thereof, is described in U.S. Pat. No. 4,076,842, incorporated herein by reference. Zeolite ZSM-35, and the conventional preparation thereof, is described in U.S. Pat. No. 4,016,245, incorporated herein by reference. Another preparation of ZSM-35 is described in U.S. Pat. No. 4,107,195, the disclosure of which is incorporated herein by reference. ZSM-48, and the conventional preparation thereof, is taught by U.S. Pat. No. 4,375,573, incorporated herein by reference. Other examples of zeolite catalysts are described in U.S. Pat. No. 5,019,663 and U.S. Pat. No. 7,022,888, also incorporated herein by reference. An exemplary condensation catalyst is a ZSM-5 zeolite modified with Cu, Pd, Ag, Pt, Ru, Re, Ni, Sn, or combinations thereof.

As described in U.S. Pat. No. 7,022,888, which is incorporated herein by reference, the condensation catalyst may be a bifunctional pentasil zeolite catalyst including at least one metallic element from the group of Cu, Ag, Au, Pt, Ni, Fe, Co, Ru, Zn, Cd, In, Rh, Pd, Ir, Re, Mn, Cr, Mo, W, Sn, Os, alloys and combinations thereof, or a modifier from the group of In, Zn, Fe, Mo, Au, Ag, Y, Sc, Ni, P, Ta, lanthanides, and combinations thereof. The zeolite may have strong acidic sites, and may be used with reactant streams containing an oxygenated hydrocarbon at a temperature of below 580° C. The bifunctional pentasil zeolite may have ZSM-5, ZSM-8 or ZSM-11 type crystal structure consisting of a large number of 5-membered oxygen-rings (i.e., pentasil rings). In one embodiment the zeolite will have a ZSM-5 type structure.

Alternatively, solid acid catalysts such as alumina modified with phosphates, chloride, silica, and other acidic oxides may be used. Also, sulfated zirconia, phosphated zirconia, titania zirconia, or tungstated zirconia may provide the necessary acidity. Re and Pt/Re catalysts are also useful for promoting condensation of oxygenates to C₅₊ hydrocarbons and/or C₅₊ mono-oxygenates. The Re is sufficiently acidic to promote acid-catalyzed condensation. In certain embodiments, acidity may also be added to activated carbon by the addition of either sulfates or phosphates.

The specific C₄₊ compounds produced will depend on various factors, including, without limitation, the type of oxygenated compounds in the reactant stream, condensation temperature, condensation pressure, the reactivity of the catalyst, and the flow rate of the reactant stream as it affects the space velocity, GHSV, LHSV, and WHSV. In certain embodiments, the reactant stream is contacted with the condensation catalyst at a WHSV that is appropriate to produce the desired hydrocarbon products. In one embodiment the WHSV is at least 0.1 grams of volatile (C₂₊O₁₋₃) oxygenates in the reactant stream per gram catalyst per hour. In another embodiment the WHSV is between 0.1 to 10.0 g/g hr, including a WHSV of 1, 2, 3, 4, 5, 6, 7, 8,9, 10 g/g hr, and increments between.

In certain embodiments the condensation reaction is carried out at a condensation temperature and a condensation pressure at which the thermodynamics of the proposed reaction are favorable. For volatile C₂₊O₁₋₃ oxygenates the reaction may be carried out at a temperature where the vapor pressure of the volatile oxygenates is at least 0.1 atm (and preferably a good deal higher). The condensation temperature will vary depending upon the specific composition of the oxygenated compounds. The condensation temperature will generally be greater than 80° C., or 100° C., or 125° C., or 150° C., or 175° C., or 200° C., or 225° C., or 250° C., and less than 500° C., or 450° C., or 425° C., or 375° C., or 325° C., or 275° C. For example, the condensation temperature may be between 80° C. to 500° C., or between 125° C. to 450° C., or between 250° C. to 425° C. The condensation pressure will generally be greater than 0 psig, or 10 psig, or 100 psig, or 200 psig, and less than 2000 psig, or 1800 psig or, or 1600 psig, or 1500 psig, or 1400 psig, or 1300 psig, or 1200 psig, or 1100 psig, or 1000 psig, or 900 psig, or 700 psig. For example, the condensation pressure may be greater than 0.1 atm, or between 0 and 1500 psig, or between 0 and 1200 psig.

C₄₊ alkanes and C₄₊ alkenes produced from acid condensation can have from 4 to 30 carbon atoms (C₄₊ alkanes and C₄₊ alkenes) and may be branched or straight chained alkanes or alkenes. The C₄₊ alkanes and C₄₊ alkenes may also include fractions of C₄₋₉, C₇₋₁₄, C₁₂₋₂₄ alkanes and alkenes, respectively, with the C₄₋₉ fraction directed to gasoline, the C₇₋₁₆ fraction directed to jet fuels, and the C₁₁₋₂₄ fraction directed to diesel fuel and other industrial applications, such as chemicals. Examples of various C₄₊ alkanes and C₄₊ alkenes include, without limitation, butane, butene, pentane, pentene, 2-methylbutane, hexane, hexene, 2-methylpentane, 3-methylpentane, 2,2-dimethylbutane, 2,3-dimethylbutane, heptane, heptene, octane, octene, 2,2,4,-trimethylpentane, 2,3-dimethyl hexane, 2,3,4-trimethylpentane, 2,3-dimethylpentane, nonane, nonene, decane, decene, undecane, undecene, dodecane, dodecene, tridecane, tridecene, tetradecane, tetradecene, pentadecane, pentadecene, hexadecane, hexadecene, heptyldecane, heptyldecene, octyldecane, octyldecene, nonyldecane, nonyldecene, eicosane, eicosene, uneicosane, uneicosene, doeicosane, doeicosene, trieicosane, trieicosene, tetraeicosane, tetraeicosene, and isomers thereof.

C₅₊ cycloalkanes and C₅₊ cycloalkenes produced from acid condensation can have from 5 to 30 carbon atoms and may be unsubstituted, mono-substituted or multi-substituted. In the case of mono-substituted and multi-substituted compounds, the substituted group may include a branched C₃₊ alkyl, a straight chain C₁₊ alkyl, a branched C₃₊ alkylene, a straight chain C₂₊ alkylene, a phenyl or a combination thereof. By way of example, at least one of the substituted groups include a branched C₃₋₁₂ alkyl, a straight chain C₁₋₁₂ alkyl, a branched C₃₋₁₂ alkylene, a straight chain C₁₋₁₂ alkylene, a straight chain C₂₋₁₂ alkylene, a phenyl or a combination thereof. By way of further example, at least one of the substituted groups include a branched C₃₋₄ alkyl, a straight chain C₁₋₄ alkyl, a branched C₁₋₄ alkylene, straight chain C₁₋₄ alkylene, straight chain C₂₋₄ alkylene, a phenyl or a combination thereof. Examples of desirable C₅₊ cycloalkanes and C₅₊ cycloalkenes include, without limitation, cyclopentane, cyclopentene, cyclohexane, cyclohexene, methyl-cyclopentane, methyl-cyclopentene, ethyl-cyclopentane, ethyl-cyclopentene, ethyl-cyclohexane, ethyl-cyclohexene, propyl-cyclohexane, butyl-cyclopentane, butyl-cyclohexane, pentyl-cyclopentane, pentyl-cyclohexane, hexyl-cyclopentane, hexyl-cyclohexane, and isomers thereof.

Aryls will generally consist of an aromatic hydrocarbon in either an unsubstituted (phenyl), mono-substituted or multi-substituted form. In the case of mono-substituted and multi-substituted compounds, the substituted group may include a branched C₃₊ alkyl, a straight chain C₁₊ alkyl, a branched C₃₊ alkylene, a straight chain C₂₊ alkylene, a phenyl or a combination thereof. By way of example, at least one of the substituted groups include a branched C₃₊ alkyl, a straight chain C₁₋₁₂ alkyl, a branched C₃₋₁₂ alkylene, a straight chain C₂₋₁₂ alkylene, a phenyl or a combination thereof. By way of further example, at least one of the substituted groups include a branched C₃₋₄ alkyl, a straight chain C₁₋₄ alkyl, a branched C₃₋₄ alkylene, straight chain C₂₋₄ alkylene, a phenyl or a combination thereof. Examples of various aryls include, without limitation, benzene, toluene, xylene (dimethylbenzene), ethyl benzene, para xylene, meta xylene, ortho xylene, C₉₊ aromatics, butyl benzene, pentyl benzene, hexyl benzene, heptyl benzene, octyl benzene, nonyl benzene, decyl benzene, undecyl benzene, and isomers thereof.

Fused aryls will generally consist of bicyclic and polycyclic aromatic hydrocarbons, in either an unsubstituted, mono-substituted, or multi-substituted form. In the case of mono-substituted and multi-substituted compounds, the substituted group may include a branched C₃₊ alkyl, a straight chain C₁₊ alkyl, a branched C₃₊ alkylene, a straight chain C₂₊ alkylene, a phenyl or a combination thereof. By way of example, at least one of the substituted groups include a branched C₃₋₄ alkyl, a straight chain C₁₋₄ alkyl, a branched C₃₋₄ alkylene, straight chain C₂₋₄ alkylene, a phenyl or a combination thereof. Examples of various fused aryls include, without limitation, naphthalene, anthracene, and isomers thereof.

Polycyclic compounds will generally consist of bicyclic and polycyclic hydrocarbons, in either an unsubstituted, mono-substituted, or multi-substituted form. Although polycyclic compounds generally include fused aryls, as used herein the polycyclic compounds generally have at least one saturated or partially saturated ring. In the case of mono-substituted and multi-substituted compounds, the substituted group may include a branched C₃₊ alkyl, a straight chain C₁₊ alkyl, a branched C₃₊ alkylene, a straight chain C₂₊ alkylene, a phenyl or a combination thereof. By way of example, at least one of the substituted groups include a branched C₃₋₄ alkyl, a straight chain C₁₋₄ alkyl, a branched C₃₋₄ alkylene, straight chain C₂₋₄ alkylene, a phenyl or a combination thereof. Examples of various fused aryls include, without limitation, tetrahydronaphthalene and decahydronaphthalene, and isomers thereof.

The C₄₊ alcohols may also be cyclic, branched or straight chained, and have from 4 to 30 carbon atoms. In general, the C₄₊ alcohols may be a compound according to the formula R¹—OH, wherein R¹ is a member selected from a branched C₄₊ alkyl, straight chain C₄₊ alkyl, a branched C₄₊ alkylene, a straight chain C₄₊ alkylene, a substituted C₅₊ cycloalkane, an unsubstituted C₅₊ cycloalkane, a substituted C₅₊ cycloalkene, an unsubstituted C₅₊ cycloalkene, an aryl, a phenyl or combinations thereof. Examples of desirable C₄₊ alcohols include, without limitation, butanol, pentanol, hexanol, heptanol, octanol, nonanol, decanol, undecanol, dodecanol, tridecanol, tetradecanol, pentadecanol, hexadecanol, heptyldecanol, octyldecanol, nonyldecanol, eicosanol, uneicosanol, doeicosanol, trieicosanol, tetraeicosanol, or isomers thereof.

In some embodiments, a condensation product stream comprising C₄₊ compounds can be fractionated into various product streams, such as gasoline, jet fuel (kerosene), diesel fuel, and aromatics. For example, the condensation product stream may be passed through a three-phase separator to separate the condensation product stream into an acid condensation gas stream, an organic stream, and an aqueous stream. The organic stream and aqueous stream can be separated by density difference, while the acid condensation gas stream comprising uncondensed gases can be recycled to the acid condensation reactor to generate additional C₄₊ compounds. In some embodiments, a gas transport device, such as a blower or compressor, can be configured in the acid condensation gas stream to control the recycle pressure. In some embodiments, an optional purge stream may also be used to control the pressure of the recycle loop in the acid condensation gas stream. In some embodiments, the aqueous stream is discarded from the process, or further processed in downstream process units.

In some embodiments, the organics stream is fractionated in a distillation column to separate the organic stream into a light product stream and a heavy product stream. In some embodiments, the distillation unit is configured to remove co-boiling contaminants for benzene, toluene, or a combination thereof.

In some embodiments, the distillation column is configured to generate a heavy stream that is free or substantially free of co-boiling non-aromatic contaminants for benzene. The distillation column may remove co-boiling nonaromatic contaminants for benzene by fractionating the organic stream into a C₆₋ stream comprising benzene, co-boiling non-aromatic contaminants for benzene, and lighter products through the light product stream. The distillation column may further fractionate the organic stream into a heavy product stream comprising C₇₊ compounds.

In some embodiments, the distillation column is configured to generate a heavy stream that is free or substantially free of co-boiling nonaromatic contaminants for toluene. The distillation column may remove co-boiling nonaromatic contaminants for toluene by fractionating the organic stream into a C₇₋ or C₈₋ stream comprising toluene, co-boiling nonaromatic contaminants for toluene, and lighter products through the light product stream. The distillation column may further fractionate the organic stream into a heavy product stream comprising C₈₊ or C₉₊ compounds.

In some embodiments, the heavy product stream is fractionated in a distillation column to separate the heavy product stream comprising C₇₊ compounds, C₈₊ compounds, or C₉+ compounds into the mixed aromatic feed stream and a heavy product stream. In some embodiments, the distillation column is configured to fractionate the heavy product stream 140 into a mixed aromatic feed stream 16 comprising C₇₊ compounds and a heavy product feed stream comprising C₁₁₊ compounds. In some embodiments, the mixed aromatic feed stream comprises C₇₊ compounds, or C₈₊ compounds, or C₉₊ compounds, or C₇₋₁₀ compounds, or C₈₋₁₀ compounds, or C₉₋₁₀ compounds.

In some embodiments, the heavy stream may be further separated for use as kerosene (e.g., C₁₁₋₁₄ as jet fuel use), diesel fuel use (e.g., C₁₂₋₂₄), and lubricants or fuel oils (e.g., C₂₅₊). Alternatively, the heavy stream may be cracked to produce addition fractions for use in gasoline, kerosene, aromatics, and/or diesel fractions.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms. As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter. As used herein, the singular forms “a”, “an”, and “the” include plural embodiments unless the context clearly dictates otherwise. The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

The present invention has been described in terms of one or more preferred embodiments, and it should be appreciated that many equivalents, alternatives, variations, and modifications, aside from those expressly stated, are possible and within the scope of the invention.

### EXAMPLES

Recycling C₄₋ Light Hydrocarbon liquids to the AC Feed gives a boost to the overall yield of liquid products of between about 2% and 5%.

Recycling C₆₋ Light Hydrocarbon liquids to the AC Feed has a slightly different effect, increasing the selectivity to aromatic species while slightly reducing the yield to liquid products.

### Example 1 Process Description

Referring to FIG. 1, cooled AC reactor effluent (1) passes to the AC Product Separator (A) where it is separated into the AC organic product (2), AC aqueous effluent (3) and a vapor phase (4). The AC organic product (2) is pumped to the Lights Column (C) by the Lights Column Feed Pump (B) for lights removal and stabilization. The AC aqueous effluent (3) is sent to the Organics Recovery Column (not shown) where as much of the organic content as possible is recovered with the remaining waste water stream sent to the Outside Battery Limits (OSBL) Waste Water Treatment.

Recovered liquids from the AC Purge Gas Compression system (5) are added to the AC organic product stream (2), to make a combined feed stream (6, Lights Column feed) to the Lights Column (C). The Lights Column stabilizes the combined feed stream, reducing the Reid Vapor Pressure (RVP) of the Lights Column Bottom stream (7) and making it suitable for further fractionation on site or transportation to another location for fractionation. In practice, the Lights Column feed can include the organic phase of the crude AC product and any hydrocarbons recovered from the AC purge gas by cooling & compression.

The overhead vapor stream (8) is condensed in the Lights Column Condenser (D), with the condensed liquids collecting in the Lights Column Reflux Drum (E). Uncondensed vapors leave in the Lights Column Vent Stream (10) and are sent to the Hydrogen plant to be added to the feed. Approximately 57% of the liquid in the Lights Column Reflux Drum (E) is returned to the Lights Column using the Lights Column Reflux Pump (F) and the Lights Column reflux stream (9), while the rest, the Light Hydrocarbon Recycle Stream (12) is pumped by the Light Hydrocarbon Recycle Pump (G) into the AC Recycle Gas stream (13), where it is vaporized and added to the Feed to the AC Reactor stream (14). In some cases, the systems and methods disclosed herein may include a Lights column reboiler (H). In some cases, the systems and methods disclosed herein may include a stream of Lights column produced water (11).

### Example 2 Light Hydrocarbon Recycle

There are two versions of the Light Hydrocarbon Recycle. The C₄₋ recycle which provides a boost to the overall yield of liquid products. The other is the C₆₋ recycle which recycles more of the Lights Column overhead liquid product back to the feed to AC. This reduces the overall yield of liquid products a little but increases the selectivity to xylenes and toluene. The Light Column is operated under different conditions for the C₄₋ vs C₆₋ recycle processes. In the C₆₋ recycle case, C₆ components leave in the overheads, whereas the C₅₊ compounds are kept in the bottoms stream during the C₄₋ recycle case. In a representative C₄₋ LHR process, the Lights Column has an overheads pressure of 165 psia (at both start of run (SOR) and end of run (EOR)) and a temperature of 67° C. (152 F, SOR) to 91° C. (196 F, EOR). The vapors are condensed and cooled to 40° C. (104 F) at both SOR & EOR, so the C₄₋ LHR is at this temperature. In comparison, the C₆₋ LHR can be carried out at the same temperature, but at a lower pressure of 80 psia.

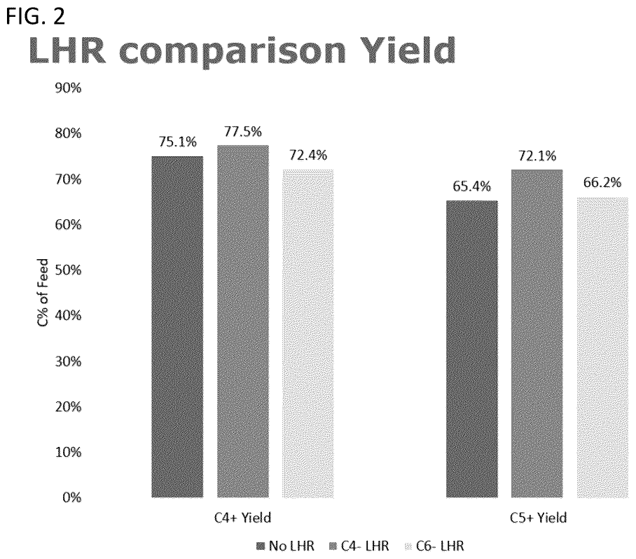

FIG. 2 illustrates the improvement that implementing a C₄₋ Light Hydrocarbon Recycle has on the yield of both C₄₊ and C₅₊ liquid products. There is an increase of 2.4% for the C₄₊ yield and an increase of 6.7% for the C₅₊ yield by using the C₄₋ Light Hydrocarbon recycle. Similarly implementing the C₆₋ Light Hydrocarbon Recycle gives a 2.7% reduction in the C₄₊ yield to liquid products, but a 0.8% increase in the C₅₊ yield.

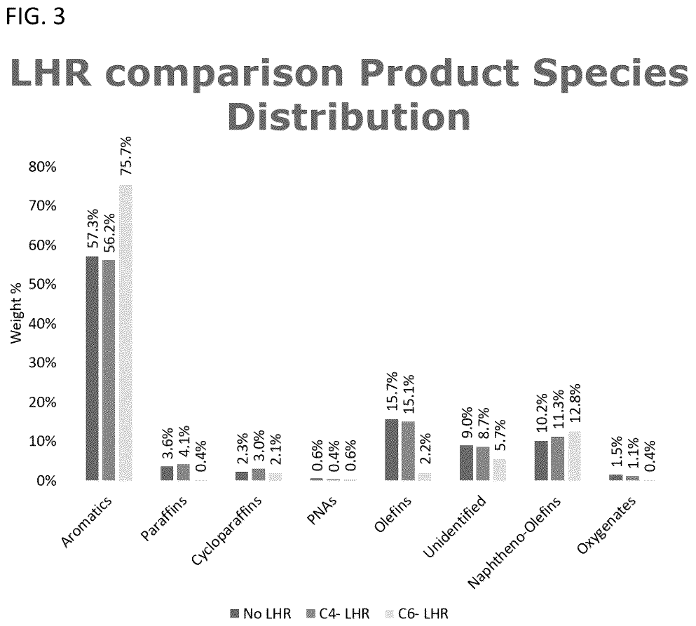

FIG. 3 shows how the C₆₋ Light Hydrocarbon Recycle gives a 18.4% increase in the wt % of aromatics compared to the no LHR case and a 19.5% increase over the C₄₋ LHR case. The C₆₋ Light Hydrocarbon Recycle also reduces the concentration of paraffins, olefins and oxygenates in the product.

What is claimed is:

1. A method of producing a C₄₊ compound, the method comprising:
(i) reacting a feed stream comprising C₁₊O₁₋₃ hydrocarbons in the presence of an acid condensation (AC) catalyst at a condensation temperature and condensation pressure to produce an AC product stream comprising the C₄₊ compound;
(ii) fractionating AC product stream into an AC liquid product stream comprising organic products and a vapor stream;
(iii) fractionating the AC liquid product stream by a fractionation column into a liquid C₄₊ compound product stream and a gaseous overhead stream;
(iv) cooling the gaseous overhead stream to produce a liquid reflux stream and a gaseous vent stream;
(v) recycling a first portion of the liquid reflux stream to the fractionation column for the fractionation in step (iii); and
(vi) recycling a second portion of the liquid reflux stream to the feed stream for the reaction in step (i).

2. The method of claim 1, wherein step (ii) is carried out in a first phase separator.

3. The method of claim 1, wherein step (iv) comprises:
(iv-a) cooling the gaseous overhead stream by a condenser to produce a cooled overhead stream; and
(iv-b) fractionating the cooled overhead stream in a second phase separator into the liquid reflux stream and the gaseous vent stream.

4. The method of claim 1, wherein a first portion of the vapor stream of step (ii) is recycled to the feed stream for the reaction in step (i).

5. The method of claim 4, wherein the first portion of the vapor stream of step (ii) is combined with the second portion of the liquid reflux stream of step (vi) to form a combined recycle stream, which is recycled to the feed stream for the reaction in step (i).

6. The method of claim 5, wherein a second portion of the vapor stream of step (ii) is condensed to produce a recovered liquid stream.

7. The method of claim 6, wherein the recovered liquid stream is combined with the AC liquid product stream of step (ii) to form a combined AC liquid product stream, which is fractionated in step (iii) to produce the liquid C₄₊ compound product stream and the gaseous overhead stream. 8 The method of claim 1, wherein the ratio of the first portion of the liquid reflux stream of step (v) to the second portion of the liquid reflux stream of step (vi) is about 40:60 to about 60:40.

9. The method of claim 1, wherein the liquid C₄₊ compound product stream has:
C₄₊ compounds at a yield of at least 75% based on total carbon amount of the feed stream;
C₅₊ compounds at a yield of at least 70% based on total carbon amount of the feed stream; and
at least 50% aromatic compounds by weight.

10. The method of claim 1, wherein the liquid C₄₊ compound product stream has:
C₄₊ compounds at a yield of at least 70% based on total carbon amount of the feed stream;
C₅₊ compounds at a yield of at least 65% based on total carbon amount of the feed stream; and
at least 70% aromatic compounds by weight.

11. The method of claim 1, wherein the acid condensation catalyst comprises carbides, nitrides, zirconia, alumina, silica, aluminosilicates, phosphates, zeolites, titanium oxides, zinc oxides, vanadium oxides, lanthanum oxides, yttrium oxides, scandium oxides, magnesium oxides, cerium oxides, barium oxides, calcium oxides, hydroxides, heteropolyacids, inorganic acids, and combinations thereof.

12. The method of claim 11, wherein the acid condensation catalyst further comprises a modifier selected from the group consisting of Ce, La, Y, Sc, P, B, Bi, Li, Na, K, Rb, Cs, Mg, Ca, Sr, Ba, and a combination thereof.

13. The method of claim 1, wherein the feed stream is derived from biomass.
